(19) United States
(12) Patent Application Publication
(10) Pub. No.: US 2025/0387158 A1
(43) Pub. Date: Dec. 25, 2025

(54) LEAFLET MODIFICATION DEVICE HAVING ROTATABLE ELECTROCAUTERY ELECTRODE

(71) Applicant: BOSTON SCIENTIFIC SCIMED, INC., MAPLE GROVE, MN (US)

(72) Inventors: Daniel Presteng, Corcoran, MN (US); Philip Andrew Litecky, Forest Lake, MN (US); Eric Michael Petersen, Maple Grove, MN (US); Joel T. Eggert, Plymouth, MN (US); Joseph Edward Adriaens, Elk River, MN (US)

(73) Assignee: BOSTON SCIENTIFIC SCIMED, INC., MAPLE GROVE, MN (US)

(21) Appl. No.: 19/241,971

(22) Filed: Jun. 18, 2025

Related U.S. Application Data
(60) Provisional application No. 63/661,691, filed on Jun. 19, 2024.

(51) Int. Cl. A61B 18/14; A61B 17/00; A61B 18/00

(52) U.S. Cl. CPC A61B 18/1492; A61B 2017/00367; A61B 2018/00178; A61B 2018/0022; A61B 2018/00369; A61B 2018/00595

(57) ABSTRACT

A medical device for lacerating valve leaflets includes an elongate shaft extending proximally from a distal region and an expandable element rotatably secured to the distal region, the expandable element is movable between a collapsed configuration and an expanded configuration. An electrocautery electrode is supported by the expandable element, the electrocautery electrode is urged in an outward direction when the expandable element is in the expanded configuration.

### CROSS-REFERENCE TO RELATED APPLICATION

This application is a continuation of U.S. Patent Application Ser. No. 63/661,691, filed Jun. 19, 2024, entitled “LEAFLET MODIFICATION DEVICE HAVING ROTATABLE ELECTROCAUTERY ELECTRODE”, which is incorporated by reference herein in its entirety.

### TECHNICAL FIELD

The present disclosure relates generally to medical devices. More particularly, the present disclosure pertains to medical devices for lacerating cardiac valve leaflets.

### BACKGROUND

A wide variety of intracorporeal medical devices have been developed for medical use, for example, intravascular use. Some of these devices include devices for lacerating cardiac valve leaflets. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

The disclosure is directed to design, material, manufacturing method, and use alternatives for lacerating cardiac valve leaflets. An example may be found in a medical device for lacerating valve leaflets. The medical device includes an elongate shaft extending proximally from a distal region and an expandable element that is rotatably secured to the distal region. The expandable element has a distal portion and a proximal portion and is movable between a collapsed configuration and an expanded configuration. An electrocautery electrode is supported by the expandable element and is urged in an outward direction when the expandable element is in the expanded configuration.

Alternatively or additionally, the distal region of the elongate shaft may include an engagement surface that is fixed relative to the elongate shaft and the proximal portion of the expandable element may include an eccentric cam surface adapted to engage the engagement surface.

Alternatively or additionally, the medical device may further include a pull wire that is coupled relative to the eccentric cam surface such that pulling on the pulling wire causes the eccentric cam surface to engage the engagement surface and causes the expandable element to rotate relative to the elongate shaft.

Alternatively or additionally, the medical device may further include a resistive element that is adapted to resist rotation of the expandable element.

Alternatively or additionally, the resistive element may allow the expandable element to rotate relative to the elongate shaft when a tensile force is applied to the pull wire and may cause the expandable element to return to an initial rotational position when the tensile force is removed from the pull wire.

Alternatively or additionally, the elongate shaft may include a symmetric multi-lumen extrusion.

Alternatively or additionally, the expandable element may include an inflatable balloon.

Alternatively or additionally, the inflatable balloon may include a proximal balloon waist and a distal balloon waist.

Alternatively or additionally, the medical device may further include a proximal fixed hub that is secured to the distal region of the elongate shaft and that includes the engagement surface. A proximal rotating hub is adapted to rotate relative to the proximal fixed hub and includes the eccentric cam surface. The proximal rotating hub is secured to the proximal balloon waist.

Alternatively or additionally, the medical device may further include one or more O-rings that are disposed between the proximal fixed hub and the proximal rotating hub.

Alternatively or additionally, the medical device may further include an electrical contact disk that rotates with the proximal rotating hub and a connector that is secured to the proximal fixed hub. The connector is electrically coupled with a conductive member extending through the elongate shaft. The electrical contact disk is electrically coupled with the electrocautery electrode.

Alternatively or additionally, the medical device may further include a distal fixed hub that is secured about a guidewire lumen extending distally from the elongate shaft and a distal rotating hub that is adapted to rotate relative to the distal fixed hub. The distal rotating hub is secured to the distal balloon waist.

Alternatively or additionally, the medical device may further include one or more O-rings disposed between the distal fixed hub and the distal rotating hub.

Another example may be found in a medical device for lacerating valve leaflets. The medical device includes an elongate shaft having a distal region and a proximal fixed hub that is secured to the distal region and that defines a fixed engagement surface. A proximal rotating hub is adapted to rotate relative to the proximal fixed hub and defines an eccentric cam surface. An inflatable balloon is secured to the proximal rotating hub and an electrocautery electrode is supported by the inflatable balloon. A pull wire is coupled to the proximal rotating hub such that pulling on the pulling wire causes the eccentric cam surface to engage the fixed engagement surface and causes the proximal rotating hub to rotate relative to the proximal fixed hub.

Alternatively or additionally, the medical device may further include a resistive element that is adapted to resist rotation of the proximal rotating hub.

Alternatively or additionally, the resistive element may allow the proximal rotating hub to rotate relative to the proximal fixed hub when a tensile force is applied to the pull wire and may cause the proximal rotating hub to return to an initial rotational position when the tensile force is removed from the pull wire.

Alternatively or additionally, the medical device may further include an electrical contact disk that rotates with the proximal rotating hub and a connector that is secured to the proximal fixed hub. The connector is electrically coupled with a conductive member extending through the elongate shaft and the electrical contact disk is electrically coupled with the electrocautery electrode.

Another example may be found in a medical device. The medical device includes an elongate shaft having a distal region, a proximal fixed hub that is secured to the distal region and that defines a fixed engagement surface, and a proximal rotating hub that is adapted to rotate relative to the proximal fixed hub. The proximal rotating hub defines an eccentric cam surface. An inflatable balloon includes a proximal balloon waist and a distal balloon waist. The proximal balloon waist is secured to the proximal rotating hub. An electrocautery electrode is supported by the inflatable balloon. A pull wire is coupled to the proximal rotating hub such that pulling on the pulling wire causes the eccentric cam surface to engage the fixed engagement surface and causes the proximal rotating hub to rotate relative to the proximal fixed hub.

Alternatively or additionally, the medical device may further include a distal fixed hub that is secured about a guidewire lumen extending distally from the elongate shaft and a distal rotating hub that is adapted to rotate relative to the distal fixed hub and is secured to the distal balloon waist.

Alternatively or additionally, the medical device may further include a resistive element that is adapted to allow the proximal rotating hub to rotate relative to the proximal fixed hub when a tensile force is applied to the pull wire and to cause the proximal rotating hub to return to an initial rotational position when the tensile force is removed from the pull wire.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cutaway view showing a replacement heart valve implant positioned within a native valve annulus of a heart;

FIG. 2 is a schematic view of an illustrative medical device;

FIG. 3 is a schematic view of the illustrative medical device of FIG. 2, shown with a control knob in a first position corresponding to the electrocautery electrode being in a first position;

FIG. 4 is a schematic view of the illustrative medical device of FIG. 2, shown with the control knob in a second position corresponding to the electrocautery electrode being in a second position;

FIG. 5 is a schematic view of a portion of the illustrative medical device of FIG. 2, corresponding to the electrocautery electrode in the first position shown in FIG. 3;

FIG. 6 is a schematic view of a portion of the illustrative medical device of FIG. 2, corresponding to the electrocautery electrode in the second position shown in FIG. 4;

FIG. 7 is a schematic view of an illustrative proximal fixed hub and an illustrative proximal rotating hub;

FIG. 8 is a schematic view showing how the illustrative proximal rotating hub of FIG. 7 rotates in response to a tensile force applied to the pull wire;

FIG. 9 is a cross-sectional view taken along line 9-9 of FIG. 2;

FIG. 10 is a cross-sectional view taken along line 10-10 of FIG. 9; and

FIG. 11 is a schematic cross-sectional view.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

A number of patients receive artificial heart valves for a variety of reasons including valve malfunction due to calcium accumulation. When an artificial heart valve is implanted, the artificial heart valve may have an expandable frame that presses the native valve leaflets away from the native position of the native valve leaflets. In some instances, the native valve is the aortic valve, and the artificial heart valve is an artificial aortic valve. In some instances, it is possible for one or more of the native valve leaflets, when pressed to the side, to at least partially or even completely block an ostium of one of the coronary arteries. Not only does this present possible health concerns for the patient, particularly if an ostium is completely blocked, but even when an ostium is only partially blocked and thus still permits blood flow, this may present difficulties in subsequently being able to perform balloon angioplasty, or place a stent, in one of the coronary arteries. In some instances, it may be beneficial to slice or lacerate with opportunity to remove one or more of the native valve leaflets prior to implantation of the artificial heart valve so that when the native valve leaflets are pressed to the side by the expandable frame of the artificial heart valve, the native valve leaflets do not block an ostium of any of the coronary arteries.

In some instances, a patient may already have an implanted artificial heart valve such as an artificial aortic valve. The artificial valve leaflets forming part of the already implanted artificial heart valve can be just as problematic with respect to potentially blocking a cardiac artery ostium when displaced to the side when a second artificial heart valve is implanted in place of the first artificial heart valve. The artificial valve leaflets forming part of the artificial heart valve may, for example, be made from porcine or bovine tissue, or may be polymeric. In some instances, artificial valve leaflets may be made of polymers such as Dacron or Gore-Tex. As discussed here, reference to a valve leaflet may refer to either a native valve leaflet or an artificial valve leaflet.

In some instances, a medical device for lacerating valve leaflets, regardless of whether they are native valve leaflets or artificial valve leaflets, may include an elongate shaft that extends proximally from a distal region, with an expandable element rotatably secured to the distal region of the elongate shaft. The expandable element may be movable between a collapsed configuration and an expanded configuration. The medical device includes an electrocautery electrode that is supported by the expandable element and is urged in an outward direction when the expandable element is in the expanded configuration.

In some cases, the distal region of the elongate shaft may include an engagement surface fixed relative to the elongate shaft and the proximal portion of the expandable element may include an eccentric cam surface adapted to engage the engagement surface. The medical device may further include a pull wire that is coupled relative to the eccentric cam surface such that pulling on the pulling wire causes the eccentric cam surface to engage the engagement surface and causes the expandable element to rotate relative to the elongate shaft. The medical device may further include a resistive element that is adapted to resist rotation of the expandable element. In some cases, the resistive element may allow the expandable element to rotate relative to the elongate shaft when a tensile force is applied to the pull wire and may cause the expandable element to return to an initial rotational position when the tensile force is removed from the pull wire. In some cases, the elongate shaft may include a symmetric multi-lumen extrusion.

The expandable element may include an inflatable balloon having a proximal balloon waist and a distal balloon waist. In some cases, the medical device may further include a proximal fixed hub that is secured to the distal region of the elongate shaft. The proximal fixed hub includes the engagement surface. A proximal rotating hub is adapted to rotate relative to the proximal fixed hub. The proximal rotating hub includes the eccentric cam surface and is secured to the proximal balloon waist. In some cases, the medical device may further include one or more O-rings that are disposed between the proximal fixed hub and the proximal rotating hub. In some cases, the medical device may further include an electrical contact disk that rotates with the proximal rotating hub and a connector that is secured to the proximal fixed hub and is electrically coupled with a conductive member extending through the elongate shaft. The connector may be a push button connector, for example. In some cases, the connector may be a brush connector, a pogo pin or a socket connection. The electrical contact disk is electrically coupled with the electrocautery electrode.

In some cases, the medical device may further include a distal fixed hub that is secured about a guidewire lumen extending distally from the elongate shaft. A distal rotating hub may be adapted to rotate relative to the distal fixed hub. The distal rotating hub may be secured to the distal balloon waist. The medical device may further include one or more O-rings that are disposed between the distal fixed hub and the distal rotating hub.

A medical device for lacerating valve leaflets may include an elongate shaft having a distal region, a proximal fixed hub secured to the distal region and defining a fixed engagement surface, and a proximal rotating hub that defines an eccentric cam surface and that is adapted to rotate relative to the proximal fixed hub. An inflatable balloon is secured to the proximal rotating hub and an electrocautery electrode is supported by the inflatable balloon. A pull wire is coupled to the proximal rotating hub such that pulling on the pulling wire causes the eccentric cam surface to engage the fixed engagement surface and causes the proximal rotating hub to rotate relative to the proximal fixed hub. In some cases, the medical device may further include a resistive element that is adapted to resist rotation of the proximal rotating hub. As an example, the resistive element may allow the proximal rotating hub to rotate relative to the proximal fixed hub when a tensile force is applied to the pull wire and may cause the proximal rotating hub to return to an initial rotational position when the tensile force is removed from the pull wire. The medical device may further include an electrical contact disk that rotates with the proximal rotating hub and a push button connector that is secured to the proximal fixed hub and electrically coupled with a conductive member extending through the elongate shaft. The electrical contact disk is electrically coupled with the electrocautery electrode.

A medical device includes an elongate shaft having a distal region, a proximal fixed hub secured to the distal region and defining a fixed engagement surface, and a proximal rotating hub adapted to rotate relative to the proximal fixed hub, the proximal rotating hub defining an eccentric cam surface. The medical device includes an inflatable balloon including a proximal balloon waist and a distal balloon waist, the proximal balloon waist secured to the proximal rotating hub. An electrocautery electrode is supported by the expandable element. A pull wire is coupled to the proximal rotating hub such that pulling on the pulling wire causes the eccentric cam surface to engage the fixed engagement surface and causes the proximal rotating hub to rotate relative to the proximal fixed hub. In some cases, the medical device may further include a distal fixed hub that is secured about a guidewire lumen extending distally from the elongate shaft and a distal rotating hub that is adapted to rotate relative to the distal fixed hub. The distal rotating hub may be secured to the distal balloon waist. In some cases, the medical device may further include a resistive element that is adapted to allow the proximal rotating hub to rotate relative to the proximal fixed hub when a tensile force is applied to the pull wire and to cause the proximal rotating hub to return to an initial rotational position when the tensile force is removed from the pull wire.

As noted, the medical devices described herein may be used in lacerating valve leaflets regardless of whether the valve leaflets are native valve leaflets or artificial valve leaflets. In some cases, the native valve leaflets may be lacerated prior to implantation of an artificial heart valve in order to avoid possible issues with one or more of the native valve leaflets from obscuring an ostium of one of the coronary arteries. Even if blood is able to flow through the ostium and into one of the coronary arteries, having the ostium even partially blocked with a native valve leaflet can potentially cause difficulties with subsequent procedures such as performing angioplasty within one of the coronary arteries or implanting a stent within one of the coronary arteries.

In some cases, a second artificial heart valve may be implanted within a previously implanted artificial heart valve. There may be a desire to lacerate one or more of the artificial valve leaflets within the previously implanted artificial heart valve before implanting the replacement artificial heart valve within the previously implanted artificial heart valve. In some cases, lacerating one or more of the artificial valve leaflets may help reduce or eliminate potential issues with the artificial valve leaflets of the previously implanted artificial heart valve interfering with operation of the replacement artificial heart valve and/or potentially blocking an ostium of one of the coronary arteries.

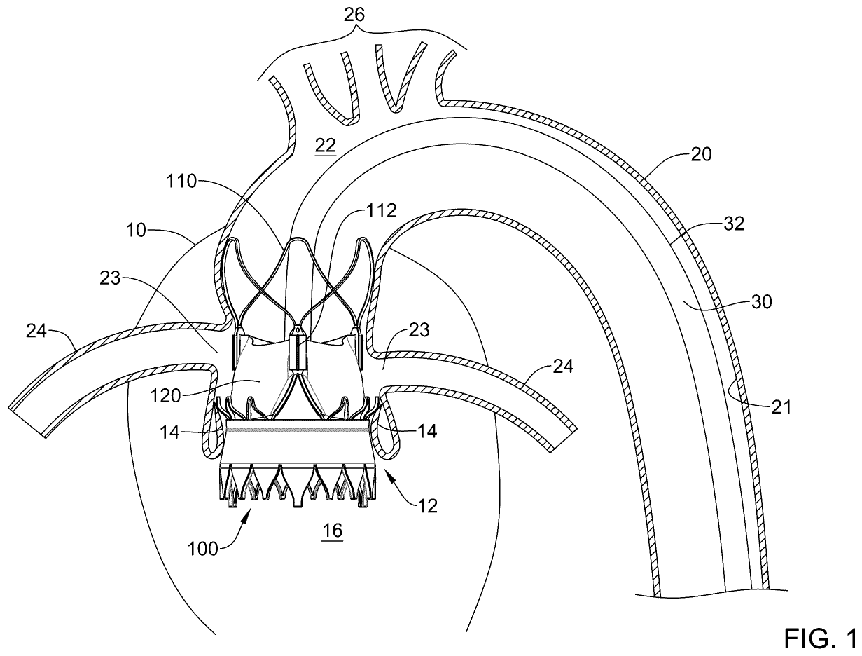

FIG. 1 is a schematic partial cut-away view of a portion of a patient's heart 10 including an aortic valve 12 having native valve leaflets 14 disposed within and/or extending from the native valve annulus, a left ventricle 16, and certain connected vasculature, such as an aorta 20 connected to the aortic valve 12 of the patient's heart 10 by an aortic arch 22 and an ascending aorta, the coronary ostia 23 of the coronary arteries 24, which extend from the aortic sinuses and/or the ascending aorta, and other large arteries 26 (e.g., subclavian and/or carotid arteries, etc.) that extend from the aortic arch 22 to important internal organs. For the purpose of this disclosure, the discussion herein is directed toward treating the aortic valve 12 and will be so described in the interest of brevity. This, however, is not intended to be limiting as the skilled person will recognize that the following discussion may also apply to other heart valves, vessels, and/or treatment locations within a patient. In some cases, the following discussion may apply to both catheter-delivered cardiac valves and surgical cardiac valves, as well as to removing problematic tissue from native anatomy.

FIG. 1 further illustrates selected aspects of a replacement heart valve implant 100 positioned within the aortic valve 12 and/or the native valve annulus of the aortic valve 12. Some non-limiting examples of the replacement heart valve 100 may include the ACURATE NEO2™ the ACURATE PRIME™, and/or family members thereof from Boston Scientific. It should be appreciated that the replacement heart valve implant 100 can be any type of replacement heart valve (e.g., a mitral valve, an aortic valve, etc.). In use, the replacement heart valve implant 100 may be implanted (e.g., such as through transcatheter delivery) in the aortic valve 12 of the heart 10. The replacement heart valve implant 100 can be configured to allow one-way flow through the replacement heart valve implant 100 from an inflow end to an outflow end.

The replacement heart valve implant 100 may include an expandable framework 110 defining a central lumen. Some suitable but non-limiting examples of materials that may be used to form the expandable framework 110, including but not limited to metals and metal alloys, composites, ceramics, polymers, and the like, are described below. The replacement heart valve implant 100 and/or the expandable framework 110 may be configured to shift between a radially collapsed configuration and a radially expanded configuration. In some instances, the expandable framework 110 may be self-expanding. In some instances, the expandable framework 110 may be self-biased toward the radially expanded configuration. In some cases, the expandable framework 110 may be mechanically expandable. As an example, the expandable framework 110 may be balloon expandable.

In some instances, the replacement heart valve implant 100 may include a plurality of valve leaflets 120 disposed within the central lumen. The plurality of valve leaflets 120 may be coupled, secured, and/or fixedly attached to the expandable framework 110 at a plurality of commissures 112. The plurality of valve leaflets 120 may be configured to shift between an open position and a closed position. The plurality of valve leaflets 120 may be configured to substantially restrict fluid flow through the replacement heart valve implant 100 in the closed position. The plurality of valve leaflets 120 may move apart from each other in the open position to permit fluid flow through the replacement heart valve implant 100.

In some cases, the plurality of valve leaflets 120 may include a polymer such as a thermoplastic polymer. In some cases, the plurality of valve leaflets 120 may include at least 50 percent by weight of a polymer. In some instances, the plurality of valve leaflets 120 may be formed from porcine pericardium, bovine pericardium, or other tissue. Other configurations and/or materials are also contemplated.

As seen in FIG. 1, a medical device 30 extends through the aortic arch 22 and into an interior of the previously implanted replacement heart valve implant 100. As will be described, the medical device 30 may be used for lacerating the valve leaflets 120 of the replacement heart valve implant 100. The medical device 30 includes an elongate shaft 32 that extends through the aortic arch 22 and in some cases contacts an interior wall 21 of the aorta 20. In some cases, contacting the interior wall 21 of the aorta 20 may help in guiding the medical device 30 into position relative to the replacement heart valve implant 100. Once the medical device 30 has been positioned relative to the replacement heart valve implant 100 (or relative to the native valve leaflets 14 if the aortic valve 12 is still intact and no replacement heart valve implant 100 was previously implanted), the medical device 30 may be used to lacerate one or more of the valve leaflets 120 (or the valve leaflets 14).

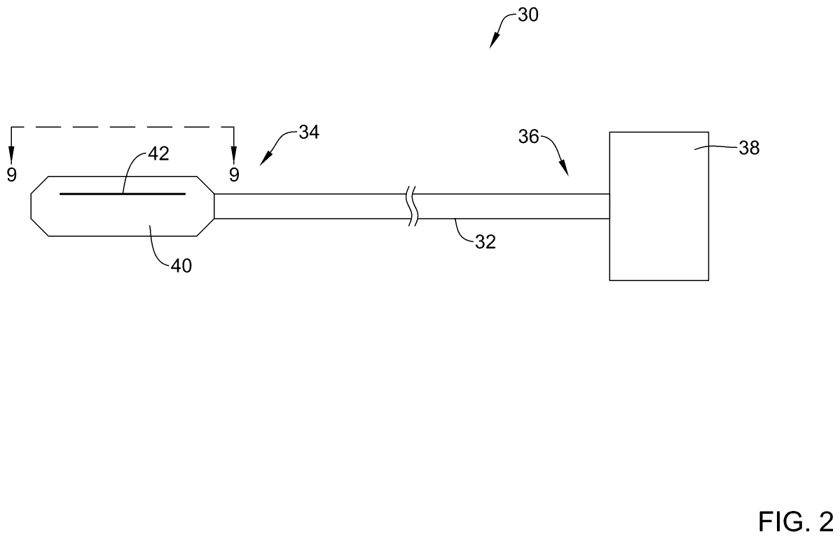

FIG. 2 is a schematic view of the medical device 30. The medical device 30 includes the elongate shaft 32. The elongate shaft 32 includes a distal region 34 and extends proximally to a proximal region 36. In some cases, the medical device 30 includes a hub 38 that is secured relative to the proximal region 36 of the elongate shaft 32. The medical device 30 includes an expandable element 40 that is secured relative to the distal region 34 of the elongate shaft 32. In some cases, the expandable element 40 may be an inflatable balloon, but this is not required in all cases. For example, the expandable element 40 may be an expandable stent. The medical device 30 includes an electrocautery electrode 42 that is disposed along an outer surface of the expandable element 40. The electrocautery electrode 42 may be used to lacerate one or more of the valve leaflets 120 (or the valve leaflets 14) when RF energy is applied to the electrocautery electrode 42.

With brief reference back to FIG. 1, it will be appreciated that positioning the medical device 30 within the aorta 20, and the aortic arch 22, may include positioning the medical device 30 such that the elongate shaft 32 is in physical contact with the interior wall 21 of the aorta 22. Having to rotate the entirety of the medical device 30 may interfere with the proper positioning of the medical device 30 relative to the valve leaflets 120 (or the valve leaflets 14). In some cases, the expandable element 40 may be able to rotate relative to the distal region 34 of the elongate shaft 32. In some cases, being able to rotate the expandable element 40 relative to the distal region 34 of the elongate shaft 32 means that the electrocautery electrode 42 may be appropriately positioned in order to lacerate a particular valve leaflet 120 (or valve leaflet 14) without having to rotate or otherwise move the elongate shaft 32. This means that the medical device 30 may be properly positioned prior to rotating the expandable element 40 to appropriately align or position the electrocautery electrode 42. Being able to rotate the expandable element 40 relative to the distal region 34 of the elongate shaft 32 also means that the expandable element 40 (and hence the electrocautery electrode 42) may be repositioned multiple times without having to move the elongate shaft 32.

While not shown in FIG. 2, the hub 38 may include one or more ports or connections to lumens extending within the elongate shaft 32. The hub 38 may accommodate a guidewire lumen extending through the hub 38 and through the elongate shaft 32. The hub 38 may accommodate an electrical connection to an electrical conductor (not shown) that extends through a lumen within the elongate 32 and that is electrically coupled with the electrocautery electrode 42. The hub 38 may accommodate a fluid coupling for providing an inflation fluid through a lumen extending within the elongate shaft 32 for inflating the expandable element 40. The hub 38 may also accommodate one or more pulling wires that may be used to cause the expandable element 40 to rotate relative to the distal region 34 of the elongate shaft 32.

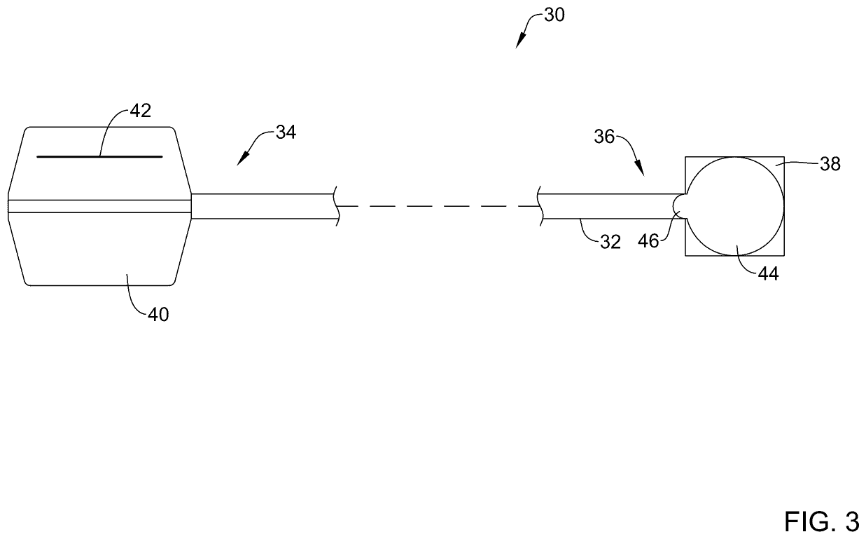

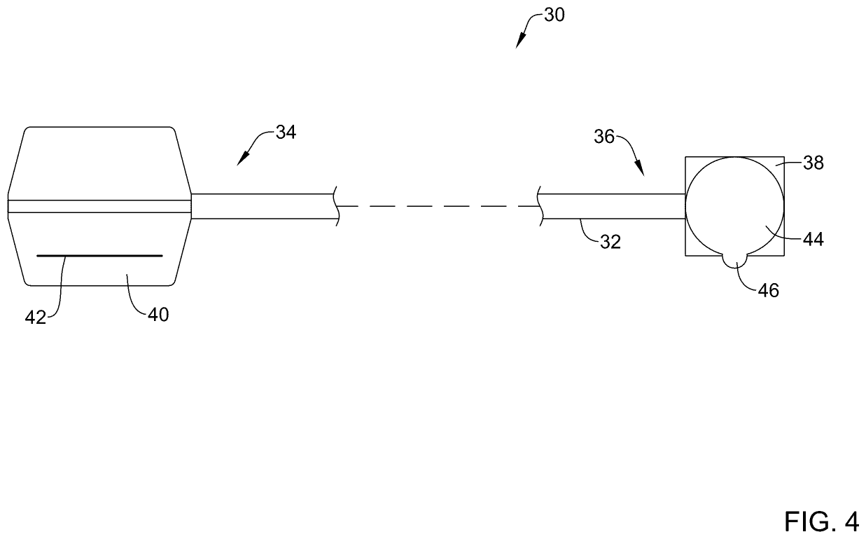

FIGS. 3 and 4 are schematic views of the medical device 30 showing how moving a control knob 44 rotates the relative position and orientation of the expandable element 40 and the electrocautery electrode 42. Rotating the control knob 44 relative to the hub 38 may actuate one or more pulling wires (not shown) that cause the expandable element 40 to rotate relative to the distal region 34 of the elongate shaft 32. The control knob 44 includes a pointer 46. The expandable element 40 is shown in FIGS. 3 and 4 as an inflatable balloon that is in its inflated or expanded configuration. Looking at FIG. 3, it can be seen that the control knob 44 has a first rotational position relative to the elongate shaft 32 in which the pointer 46 is aligned with the elongate shaft 32. This corresponds to the electrocautery electrode 42 being in a first rotational position (referring to rotation of the expandable element 40) in which the electrocautery electrode 42 is shown as being on an upper (in the illustrated orientation) portion of the expandable element 40. In FIG. 4, the control knob 44 has been rotated to a second rotational position relative to the elongate shaft 32 in which the pointer 46 is perpendicular to the elongate shaft 32. This corresponds to the electrocautery electrode 42 being in a second rotational position (referring to rotation of the expandable element 40) in which the electrocautery electrode 42 is shown as being on a lower (in the illustrated orientation) portion of the expandable element 40.

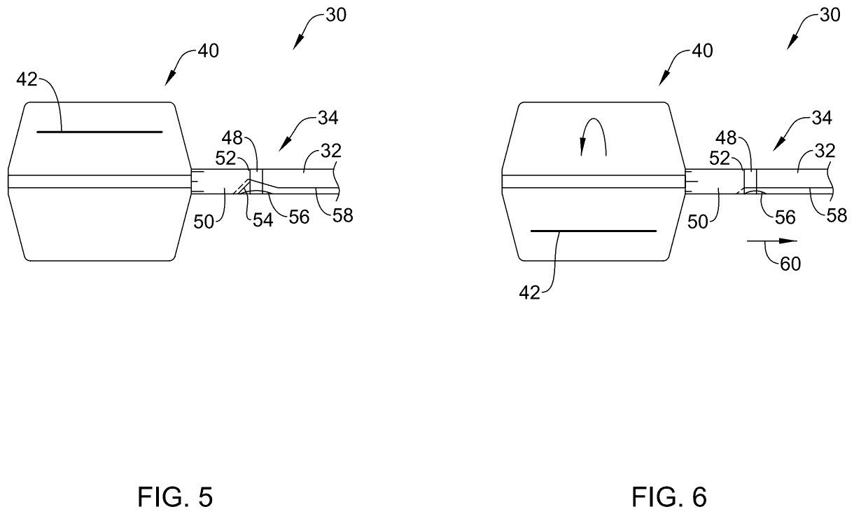

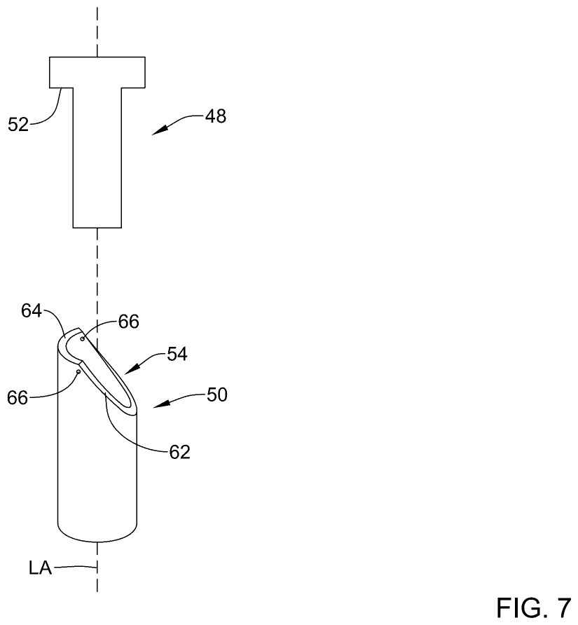

FIGS. 5 and 6 provide additional details regarding the connection between the expandable element 40 and the distal region 34 of the elongate shaft 32, including an example of how the expandable element 40 may be rotated relative to the distal region 34 of the elongate shaft 32 in order to control the relative position of the electrocautery electrode 42. It will be appreciated that FIG. 5 corresponds to the expandable element 40 being positioned to place the electrocautery electrode 42 in the first rotational position shown in FIG. 3 while FIG. 6 corresponds to the expandable element 40 being positioned to place the electrocautery electrode 42 in the second rotational position shown in FIG. 4. FIG. 7 may be considered as being an exploded view of the elements that contribute to being able to rotate the expandable element 40 relative to the distal region 34 of the elongate shaft 32.

A proximal fixed hub 48 is secured relative to the distal region 34 of the elongate shaft 32. A proximal rotating hub 50 is positioned over the proximal fixed hub 48 such that the proximal rotating hub 50 is able to rotate relative to the proximal fixed hub 48. The proximal rotating hub 50 may be secured to the expandable element 40 such that the expandable element 40 rotates relative to the elongate shaft 32 when the proximal rotating hub 50 rotates relative to the proximal fixed hub 48.

The proximal fixed hub 48 defines or otherwise includes an engagement surface 52 that is adapted to engage with an eccentric cam surface 54 that forms a proximal surface of the proximal fixed hub 48. A resistive element 56 is positioned to resist movement of the proximal rotating hub 50 relative to the proximal fixed hub 48. A pull wire 58 extends proximally from the proximal rotating hub 50. By pulling the pull wire 58 in a direction indicated by an arrow 60 (see FIG. 6), the proximal rotating hub 50 is pulled towards the proximal fixed hub 48 such that the eccentric cam surface 54 engages the engagement surface 52 of the proximal fixed hub 48. The interaction between the eccentric cam surface 54 of the proximal rotating hub 50 and the engagement surface 52 of the proximal fixed hub 48 causes the proximal rotating hub 50 to rotate relative to the proximal fixed hub 48, thereby changing the relative position of the electrocautery electrode 42. As best seen in FIG. 7, the eccentric cam surface 54 of the proximal rotating hub 50 includes a first portion 62 that defines an angle that may range from about 30 degrees to about 60 degrees relative to a longitudinal axis LA. As an example, the first portion 62 may define an angle of about 45 degrees with respect to the longitudinal axis LA. The eccentric cam surface 54 also includes a second portion 64 that contacts the engagement surface 52 of the proximal fixed hub 48, allowing the medical device 30 to maintain a consistent positioning depth during rotation.

As noted, the resistive element 56 resists movement of the proximal rotating hub 50 relative to the proximal fixed hub 48, although applying a tensile force via the pull wire 58 can overcome this resistance. In some cases, relaxing the tensile force applied to the pull wire 58 will cause the resistive element 56 to cause the proximal rotating hub 50 to return to its initial position relative to the proximal fixed hub 48. In other words, pulling the pull wire 58 causes the proximal rotating hub 50 to rotate relative to the proximal fixed hub 48, causing the expandable element 40 (and thus the electrocautery electrode 42) to move from its first position (shown in FIGS. 3 and 5) towards its second position (shown in FIGS. 4 and 6). In some cases, releasing the pull wire 58 causes the proximal rotating hub 50 to rotate back relative to the proximal fixed hub 48, causing the expandable element 40 (and thus the electrocautery electrode 42) to move from its second position (shown in FIGS. 4 and 6) back towards its first position (shown in FIGS. 3 and 5).

While a single pull wire 58 is shown, in some cases the medical device 30 may include two pull wires 58, such that pulling one of the pull wires 58 causes the proximal rotating hub 50 to rotate in a first rotational direction relative to the proximal fixed hub 48 and pulling the other one of the pull wires 58 causes the proximal rotating hub 50 to rotate in a second rotational direction (opposite that of the first rotational direction) relative to the proximal fixed hub 48. In either case, the resistive element 56 may return the proximal rotating hub 50 (and hence the expandable element 40) back to a starting position. In some cases, the resistive element 56 may be formed of spring steel. FIG. 7 shows a pair of apertures 66 to which each of a pair of pull wires 58 may be secured.

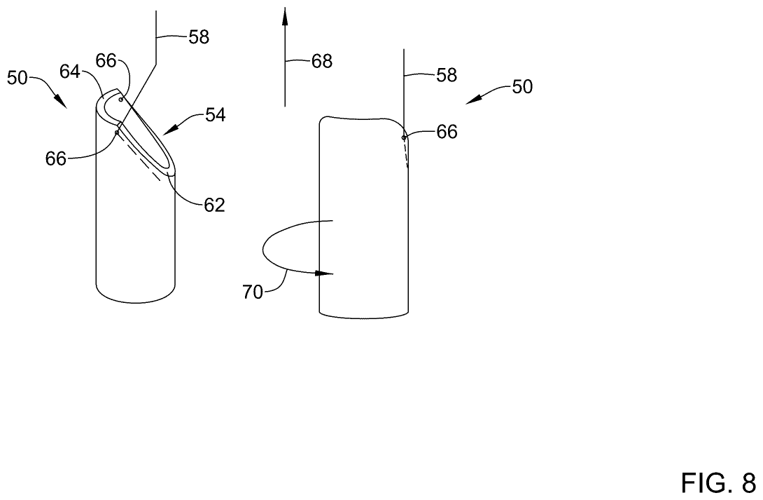

FIG. 8 is a schematic view showing how the illustrative proximal rotating hub 50 rotates in response to a tensile force applied to the pull wire 58. On the left side of FIG. 8 is a view of the proximal rotating hub 50 before any tensile force is applied to the pull wire 58. When a tensile force is applied to the pull wire 58 (in a direction indicated by an arrow 68), the eccentric cam surface 54 engages the engagement surface 52 (see FIG. 7). This causes the proximal rotating hub 50 to rotate relative to the proximal fixed hub 48 (See FIG. 7) in a direction indicated by an arrow 70.

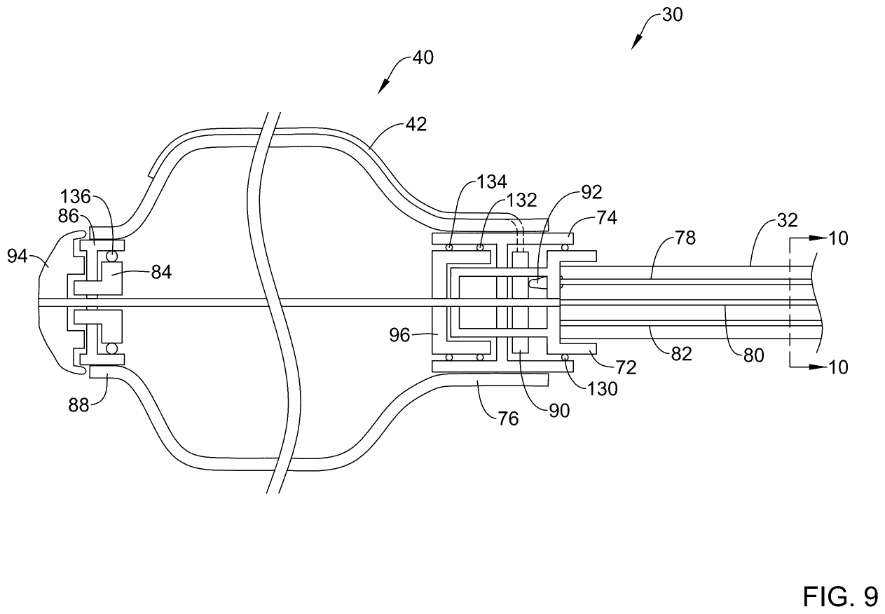

FIG. 9 is a cross-sectional view taken along line 9-9 of FIG. 2, providing an example of allowing the expandable element 40 to rotate relative to the distal region 34 of the elongate shaft 32. As shown, the medical device 30 includes a proximal fixed hub 72 that is fixedly secured to the distal region 34 of the elongate shaft 32. The proximal fixed hub 72 may be considered as being an example of the proximal fixed hub 48. A proximal rotating hub 74, which may be considered as being an example of the proximal rotating hub 50, is disposed over the proximal fixed hub 72 and is able to rotate relative to the proximal fixed hub 72. A proximal waist 76 of the expandable element 40 is secured to the proximal fixed hub 72 such that the expandable element 40 rotates relative to the proximal fixed hub 72 when the proximal rotating hub 74 rotates relative to the proximal fixed hub 72. The elongate shaft 32 defines several internal lumens, including an electrical conductor lumen 78 that is adapted to accommodate an electrical conductor extending therethrough, a guidewire lumen 80 that as shown may extend distally through the expandable element 40, and an inflation lumen 82 that is fluidly coupled with an interior of the expandable element 40 in order to expand the expandable element 40.

The medical device 30 includes a distal fixed hub 84 that is secured relative to the guidewire lumen 82 and a distal rotating hub 86 that is disposed about the distal fixed hub 84 and is adapted to rotate relative to the distal fixed hub 84. Thus, when the expandable element 40 is urged into rotation by the proximal rotating hub 74 rotating relative to the proximal fixed hub 72, the combination of the distal fixed hub 84 secured relative to the guidewire lumen 80 and the distal rotating hub 86 being free to rotate relative to the distal fixed hub 84 allows the expandable element 40 to rotate. A distal waist 88 is secured to the distal rotating hub 86.

The medical device 30 includes an electrical contact disk 90 that is adapted to spin with the proximal rotating hub 74. The electrical contact disk 90 is in electrical contact with the electrocautery electrode 42. The electrical contact disk 90 is also in electrical contact with a push button connector 92 that is electrically coupled with an electrical conductor (not shown) extending through the electrical conductor lumen 78. Thus, it is possible to make electrical connection with the electrocautery electrode 42 while allowing the expandable element 40 (and thus the electrocautery electrode 42) to rotate relative to the distal region 34 of the elongate shaft 32.

In some cases, the medical device 30 may include a distal cap 94 that provides an atraumatic tip and that rotates with the distal rotating hub 86. In some cases, the medical device 30 may include an additional component 96 that is secured to the proximal fixed hub 72. The medical device 30 may include several O-rings that help to provide a fluid-tight seal between fixed components and rotating components. For example, the medical device 30 may include an O-ring 130 that is positioned between the proximal fixed hub 72 and the proximal rotating hub 74. The medical device 30 may include an O-ring 132 and an O-ring 134 that are each positioned between the additional component 96 and the proximal rotating hub 74. The medical device 30 may include an O-ring 136 that is positioned between the distal fixed hub 84 and the distal rotating hub 86. While not expressly shown, the engagement surface 52 (see FIG. 7) may be formed as part of the proximal fixed hub 72 and the eccentric cam surface 54 (see FIG. 7) may be formed as part of the proximal rotating hub 74.

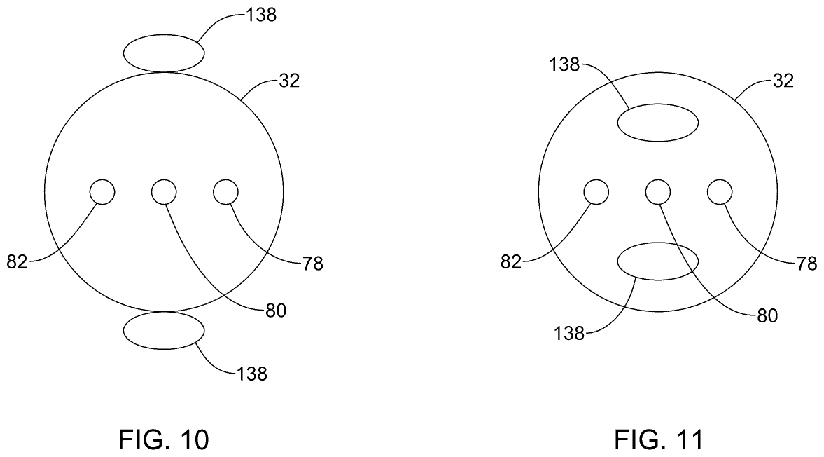

In some cases, the elongate shaft 32 may be considered as being a symmetric multi-lumen extrusion. FIG. 10 is a cross-sectional view taken along line 10-10 of FIG. 9. As can be seen, the electrical conductor lumen 78, the guidewire lumen 80 and the inflation lumen 82 are arranged symmetrically. Moreover, each of the electrical conductor lumen 78, the guidewire lumen 80 and the inflation lumen 82 can be seen as being arranged along an imaginary line transecting the elongate shaft 32. The elongate shaft 32 may also include a pair of pull wire lumens 138. In some cases, as shown in FIG. 10, the pull wire lumens 138 are outside of the elongate shaft 32. In FIG. 11, which may be considered as being a schematic cross-sectional view, the pull wire lumens 138 are located within the elongate shaft 32. In either case, an imaginary line transecting the elongate shaft 32 and both pull wire lumens 138 may be considered as orthogonal to the imaginary line extending through each of the electrical conductor lumen 78, the guidewire lumen 80 and the inflation lumen 82. In some cases, the elongate shaft 32 may be extruded to include additional lumens (not shown), but the elongate shaft 32 will remain symmetric as having a symmetric elongate shaft 32 can help to reduce or even eliminate whipping when manipulating the medical device 30.

The materials that can be used for the various components of the devices and various elements thereof disclosed herein may include those commonly associated with medical devices. In some instances, the medical devices, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof, or any other suitable material.

In at least some instances, portions or all of the medical devices described herein, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the medical devices and/or other elements disclosed herein. For example, the medical devices, and/or components or portions thereof, may be made of a material that does not substantially distort the image and create substantial artifacts (e.g., gaps in the image). Certain ferromagnetic materials, for example, may not be suitable because they may create artifacts in an MRI image. The medical assembly 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the medical devices and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical device for lacerating valve leaflets, the medical device comprising:
an elongate shaft extending proximally from a distal region;
an expandable element rotatably secured to the distal region, the expandable element having a distal portion and a proximal portion, the expandable element is movable between a collapsed configuration and an expanded configuration; and
an electrocautery electrode supported by the expandable element, the electrocautery electrode is urged in an outward direction when the expandable element is in the expanded configuration.

2. The medical device of claim 1, wherein:
the distal region of the elongate shaft includes an engagement surface fixed relative to the elongate shaft;
the proximal portion of the expandable element includes an eccentric cam surface adapted to engage the engagement surface.

3. The medical device of claim 2, further comprising a pull wire that is coupled relative to the eccentric cam surface such that pulling on the pulling wire causes the eccentric cam surface to engage the engagement surface and causes the expandable element to rotate relative to the elongate shaft.

4. The medical device of claim 3, further comprising a resistive element that is adapted to resist rotation of the expandable element.

5. The medical device of claim 4, wherein the resistive element allows the expandable element to rotate relative to the elongate shaft when a tensile force is applied to the pull wire and causes the expandable element to return to an initial rotational position when the tensile force is removed from the pull wire.

6. The medical device of claim 1, wherein the elongate shaft comprises a symmetric multi-lumen extrusion.

7. The medical device of claim 1, wherein the expandable element comprises an inflatable balloon.

8. The medical device of claim 7, wherein the inflatable balloon includes a proximal balloon waist and a distal balloon waist.

9. The medical device of claim 8, further comprising:
a proximal fixed hub secured to the distal region of the elongate shaft, the proximal fixed hub including the engagement surface; and
a proximal rotating hub adapted to rotate relative to the proximal fixed hub, the proximal rotating hub including the eccentric cam surface, the proximal rotating hub secured to the proximal balloon waist.

10. The medical device of claim 9, further comprising one or more O-rings disposed between the proximal fixed hub and the proximal rotating hub.

11. The medical device of claim 9, further comprising:
an electrical contact disk that rotates with the proximal rotating hub; and
a connector secured to the proximal fixed hub, the connector is electrically coupled with a conductive member extending through the elongate shaft;
wherein the electrical contact disk is electrically coupled with the electrocautery electrode.

12. The medical device of claim 8, further comprising:
a distal fixed hub secured about a guidewire lumen extending distally from the elongate shaft; and
a distal rotating hub adapted to rotate relative to the distal fixed hub, the distal rotating hub is secured to the distal balloon waist.

13. The medical device of claim 12, further comprising one or more O-rings disposed between the distal fixed hub and the distal rotating hub.

14. A medical device for lacerating valve leaflets, the medical device comprising:
an elongate shaft having a distal region;
a proximal fixed hub secured to the distal region and defining a fixed engagement surface;
a proximal rotating hub adapted to rotate relative to the proximal fixed hub, the proximal rotating hub defining an eccentric cam surface;
an inflatable balloon secured to the proximal rotating hub;
an electrocautery electrode supported by the inflatable balloon; and
a pull wire coupled to the proximal rotating hub such that pulling on the pulling wire causes the eccentric cam surface to engage the fixed engagement surface and causes the proximal rotating hub to rotate relative to the proximal fixed hub.

15. The medical device of claim 14, further comprising a resistive element that is adapted to resist rotation of the proximal rotating hub.

16. The medical device of claim 15, wherein the resistive element allows the proximal rotating hub to rotate relative to the proximal fixed hub when a tensile force is applied to the pull wire and causes the proximal rotating hub to return to an initial rotational position when the tensile force is removed from the pull wire.

17. The medical device of claim 14, further comprising:
an electrical contact disk that rotates with the proximal rotating hub; and
a connector secured to the proximal fixed hub, the connector is electrically coupled with a conductive member extending through the elongate shaft;
wherein the electrical contact disk is electrically coupled with the electrocautery electrode.

18. A medical device, comprising:
an elongate shaft having a distal region;
a proximal fixed hub secured to the distal region and defining a fixed engagement surface;
a proximal rotating hub adapted to rotate relative to the proximal fixed hub, the proximal rotating hub defining an eccentric cam surface;
an inflatable balloon including a proximal balloon waist and a distal balloon waist, the proximal balloon waist secured to the proximal rotating hub;
an electrocautery electrode supported by the inflatable balloon; and
a pull wire coupled to the proximal rotating hub such that pulling on the pulling wire causes the eccentric cam surface to engage the fixed engagement surface and causes the proximal rotating hub to rotate relative to the proximal fixed hub.

19. The medical device of claim 18, further comprising:
a distal fixed hub secured about a guidewire lumen extending distally from the elongate shaft; and
a distal rotating hub adapted to rotate relative to the distal fixed hub, the distal rotating hub is secured to the distal balloon waist.

20. The medical device of claim 18, further comprising a resistive element that is adapted to allow the proximal rotating hub to rotate relative to the proximal fixed hub when a tensile force is applied to the pull wire and to cause the proximal rotating hub to return to an initial rotational position when the tensile force is removed from the pull wire.
